(19) United States
(12) Patent Application Publication
(10) Pub. No.: US 2025/0186259 A1
(43) Pub. Date: Jun. 12, 2025

(54) FEMTOSECOND LASER FOR OPHTHALMIC SURGERY EMPLOYING A RESONANT SCANNER WITH IMPROVED UNIFORMITY OF LASER SPOT DISTRIBUTION

(71) Applicant: AMO Development, LLC, Irvine, CA (US)

(72) Inventors: Mohammad Saidur Rahaman, San Jose, CA (US); Pavel Vodkin, San Jose, CA (US); Rongwei Xuan, Fremont, CA (US); Yang Te Fan, Cupertino, CA (US); Hong Fu, Pleasanton, CA (US)

(21) Appl. No.: 18/929,398

(22) Filed: Oct. 28, 2024

Related U.S. Application Data
(60) Provisional application No. 63/607,260, filed on Dec. 7, 2023.

(51) Int. Cl. A61F 9/008; A61B 18/20

(52) U.S. Cl. CPC A61F 9/0084; A61B 2018/20353; A61F 2009/00897

(57) ABSTRACT

In a femtosecond ophthalmic laser system which employs a high frequency resonant scanner to produce a laser scanline and XY and Z scanners to move the scanline in a patient's eye to perform eye surgery, a beam blocking member is placed near an internal focus plane of the optical system to block some of the beam paths to truncate the laser scanline at the two ends. This eliminates the closely spaced or overlapping laser focus spots near the ends of the scanline. The beam blocking member has a plate shape with one or more apertures of different shapes or sizes, and is movable in the transverse direction to different positions to block different amounts of the scanline.

### CROSS-REFERENCE TO RELATED APPLICATIONS

The present application claims priority to U.S. Provisional Application No. 63/607,260, filed on Dec. 7, 2023, the contents of which are expressly incorporated herein by reference.

### BACKGROUND OF THE INVENTION

This invention relates to ophthalmic laser systems, and in particular, it relates to a femtosecond ophthalmic laser system using a resonant scanner.

Femtosecond laser systems are used to perform laser-assisted ophthalmic surgeries by making incisions in eye tissues such as the cornea. For a femtosecond laser using very high pulse repetition rates, e.g., 5-20 MHz, a fast scanning system is required to distribute the focus spot of the laser pulses to form laser cutting patterns in the eye tissue. Two types of high-speed scanning systems have been described. One type of system uses a multi-facet polygon mirror rotating at a high speed. This scanning method requires the use of a bulky high-speed motor supported by a ball bearing rotary spindle, which can induce system vibration. Moreover, the multi-facet polygon mirror may introduce different amount of wavefront aberrations to the laser beam, causing variations among different scanlines produced by the different facets.

Another type of scanning system uses a high speed resonant scanning mirror to produce the fast scanlines. One such system is described in commonly owned U.S. Pat. Appl. Pub. Nos. US20160374857 and US20190110926. The frequency of the resonant scanning mirror is typically between 0.5 kHz and 20 kHz, for example, between 7 kHz and 9 kHz. By applying different voltages to the resonant scanning mirror, different scanline lengths can be obtained. For example, 400 μm, 600 μm, and 900 μm scanlines may be used for different tissue incision segments. The resonant scanning mirror is a mechanical oscillator that is reactionless, thus eliminating all external vibration, and it has no wearing parts. It has a proven long operation lifetime of several billion cycles of continuous scanning (equivalent of ten years or more of useful life under normal conditions).

### SUMMARY OF THE INVENTION

The present invention is directed to a femtosecond ophthalmic laser system that employs a resonant scanner which produces improved uniformity of laser spot distribution in the scanline.

Additional features and advantages of the invention will be set forth in the descriptions that follow and in part will be apparent from the description, or may be learned by practice of the invention. The objectives and other advantages of the invention will be realized and attained by the structure particularly pointed out in the written description and claims thereof as well as the appended drawings.

To achieve the above objects, the present invention provides an ophthalmic laser system which includes: a laser device configured to generate a pulsed laser beam having a plurality of laser pulses; a high frequency scanner configured to scan the laser beam back and forth at a predefined frequency to form a plurality of scanned laser beams at different angles; a first set of optical elements and a second set of optical elements, each set of optical elements including one or more lenses, configured to focus the scanned laser beams through an internal focal plane located between the first and second sets of optical elements to a plurality of external focus spots which form a laser scanline; and a beam blocking member located in a vicinity of the internal focal plane, wherein the beam blocking member has a plate shape and defines one or more apertures; a mechanical support and movement structure configured to support and move the beam blocking member in a transverse direction perpendicular to an optical axis of the first and second set of optical elements, wherein the one or more apertures of the beam blocking member are configured to block a portion of the plurality of scanned laser beams to eliminate a portion of the external focus spots at two ends of the laser scanline, and the beam blocking member is configured to be moved in the transverse direction to different positions to block different portions of the plurality of scanned laser beams.

In another aspect, the present invention provides a method implemented in an ophthalmic laser system, which includes: by a laser device, generating a pulsed laser beam having a plurality of laser pulses; by a high frequency scanner, scanning the laser beam back and forth at a predefined frequency to form a plurality of scanned laser beams at different angles; by a first set of optical elements and a second set of optical elements, each set of optical elements including one or more lenses, focusing the scanned laser beams through an internal focal plane located between the first and second sets of optical elements to a plurality of external focus spots which form a laser scanline; by a beam blocking member located in a vicinity of the internal focal plane, blocking a portion of the plurality of scanned laser beams to eliminate a portion of the external focus spots at two ends of the laser scanline, wherein the beam blocking member has a plate shape and defines one or more apertures; and by a mechanical support and movement structure, supporting and moving the beam blocking member in a transverse direction perpendicular to an optical axis of the first and second set of optical elements to different positions to block different portions of the plurality of scanned laser beams.

It is to be understood that both the foregoing general description and the following detailed description are exemplary and explanatory and are intended to provide further explanation of the invention as claimed.

### BRIEF DESCRIPTION OF DRAWINGS

FIGS. 1A-1C schematically illustrate a portion of an optical system of an ophthalmic laser system and the principle of scanline truncation (blocking).

FIGS. 2A and 2B schematically illustrate a portion of an optical system of an ophthalmic laser system with a wedge shaped beam blocking member.

FIGS. 3A-3F illustrate examples of the shape of the beam blocking member in the plan view.

FIG. 4 schematically illustrates another method to control photodisruption at the two ends of a scanline according to an alternative embodiment of the present invention.

FIG. 5 schematically illustrates yet another method to control photodisruption at the two ends of a scanline according to another alternative embodiment of the present invention.

FIG. 6 is a block diagram illustrating a surgical ophthalmic laser system in which embodiments of the present invention may be implemented.

FIG. 7 is another diagram illustrating a surgical ophthalmic laser system in which embodiments of the present invention may be implemented.

### DETAILED DESCRIPTION OF THE INVENTION

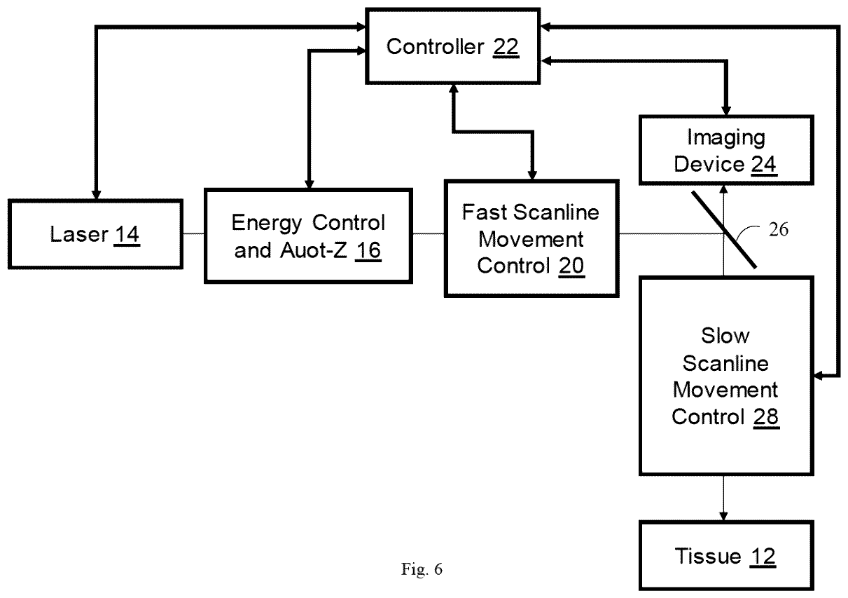

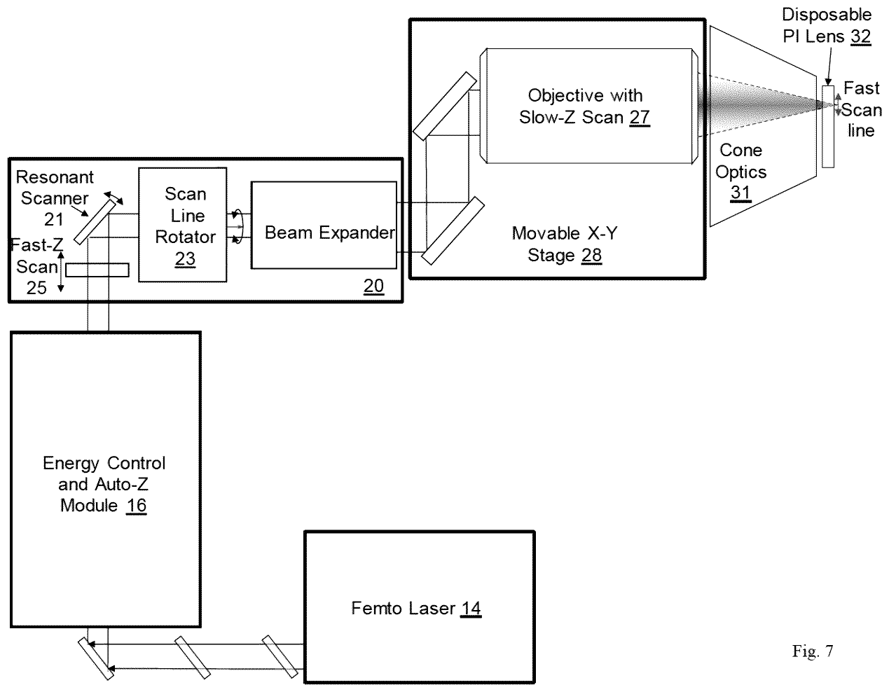

FIGS. 6 and 7 illustrate a surgical ophthalmic laser system in which embodiments of the present invention may be implemented. As shown in FIG. 6, a system 10 for making an incision in a tissue 12 of a patient's eye includes, but is not limited to, a laser 14 capable of generating a pulsed laser beam, an energy control module 16 for varying the pulse energy of the pulsed laser beam, a fast scanline movement control module 20 for generating a fast scanline of the pulsed laser beam (described in more detail later), a controller 22, and a slow scanline movement control module 28 for moving the laser scanline and delivering it to the tissue 12. The controller 22, such as a processor operating suitable control software, is operatively coupled with the fast scanline movement control module 20, the slow scanline movement control module 28, and the energy control module 16 to direct the scanline of the pulsed laser beam along a scan pattern on or in the tissue 12. In this embodiment, the system 10 further includes a beam splitter 26 and an imaging device 24 coupled to the controller 22 for a feedback control mechanism (not shown) of the pulsed laser beam. Other feedback methods may also be used. In an embodiment, the pattern of pulses may be summarized in machine readable data of tangible storage media in the form of a treatment table. The treatment table may be adjusted according to feedback input into the controller 22 from an automated image analysis system in response to feedback data provided from a monitoring system feedback system (not shown).

Laser 14 may comprise a femtosecond laser capable of providing pulsed laser beams, which may be used in optical procedures, such as localized photodisruption (e.g., laser induced optical breakdown). Localized photodisruptions can be placed at or below the surface of the tissue or other material to produce high-precision material processing. For example, a micro-optics scanning system may be used to scan the pulsed laser beam to produce an incision in the material, create a flap of the material, create a pocket within the material, form removable structures of the material, and the like. The term “scan” or “scanning” refers to the movement of the focal point of the pulsed laser beam along a desired path or in a desired pattern.

In other embodiments, the laser 14 may comprise a laser source configured to deliver an ultraviolet laser beam comprising a plurality of ultraviolet laser pulses capable of photodecomposing one or more intraocular targets within the eye.

Although the laser system 10 may be used to photoalter a variety of materials (e.g., organic, inorganic, or a combination thereof), the laser system 10 is suitable for ophthalmic applications in some embodiments. In these cases, the focusing optics direct the pulsed laser beam toward an eye (for example, onto or into a cornea) for plasma mediated (for example, non-UV) photoablation of superficial tissue, or into the stroma of the cornea for intrastromal photodisruption of tissue. In these embodiments, the surgical laser system 10 may also include a lens to change the shape (for example, flatten or curve) of the cornea prior to scanning the pulsed laser beam toward the eye.

FIG. 7 shows another exemplary diagram of the laser system 10. FIG. 7 shows components of a laser delivery system including a moveable XY-scanner (or movable XY-stage) 28 of a miniaturized femtosecond laser system. In this embodiment, the system 10 uses a femtosecond oscillator, or a fiber oscillator-based low energy laser. This allows the laser to be made much smaller. The laser-tissue interaction is in the low-density-plasma mode. An exemplary set of laser parameters for such lasers include pulse energy in the 40-100 nJ range and pulse repetitive rates (or “rep rates”) in the 2-40 MHZ range. A fast-Z scanner 25 and a resonant scanner 21 direct the laser beam to a scanline rotator 23. When used in an ophthalmic procedure, the system 10 also includes a patient interface design that has a fixed cone nose 31 and a contact lens 32 that engages with the patient's eye. A beam splitter may be placed inside the cone 31 of the patient interface to allow the whole eye to be imaged via visualization optics. In some embodiments, the system 10 may use: optics with a 0.6 numerical aperture (NA) which would produce 1.1 μm Full Width at Half Maximum (FWHM) focus spot size; and a resonant scanner 21 that produces 0.2-1.2 mm scanline with the XY-scanner scanning the resonant scanline to a 1.0 mm field. The prism 23 (e.g., a Dove or Pechan prism, or the like) rotates the resonant scanline in any direction on the XY plane. The fast-Z scanner 25 sets the incision depth. The slow scanline movement control module employs a movable XY-stage 28 carrying an objective lens with Z-scanning capability 27, referred to as slow-Z scanner because it is slower than the fast-Z scanner 25. The movable XY-stage 28 moves the objective lens to achieve scanning of the laser scanline in the X and Y directions. The objective lens changes the depth of the laser scanline in the tissue. The energy control and auto-Z module 16 may include appropriate components to control the laser pulse energy, including attenuators, etc. It may also include an auto-Z module which employs a confocal or non-confocal imaging system to provide a depth reference. The miniaturized femtosecond laser system 10 may be a desktop system so that the patient sits upright while being under treatment. This eliminates the need of certain opto-mechanical arm mechanism(s), and greatly reduces the complexity, size, and weight of the laser system. Alternatively, the miniaturized laser system may be designed as a conventional femtosecond laser system, where the patient is treated while lying down.

Using the above-described system, the beam scanning can be realized with a “fast-scan-slow-sweep” scanning scheme, also referred herein as a fast scanline scheme. The scheme consists of two scanning mechanisms: first, a high frequency fast scanner (e.g., a resonant scanner 21 of FIG. 7) is used to scan the beam back and forth to produce a short, fast scanline; second, the fast scanline is slowly swept by much slower X, Y, and Z scan mechanisms (e.g. the moveable X-Y stage 28 and the objective lens with slow-Z scan 27, and the fast-Z scanner 25). In some examples, the laser system 10 uses an 8 kHz (e.g. between 7 kHz and 9 kHz, or more generally, between 0.5 kHz and 20 kHz) resonant scanner 21 to produce a fast scanline of about 1 mm (e.g., between 0.9 mm and 1.1 mm, or more generally, between 0.2 mm and 1.2 mm) and a scan speed of about 25 m/sec, and X, Y, and Z scan mechanisms with the scan speed (sweeping speed) smaller than about 0.1 m/sec. The fast scanline may be perpendicular to the optical beam propagation direction, i.e., it is parallel to the XY plane. The trajectory of the slow sweep can be any three dimensional curve drawn by the X, Y, and Z scanning devices (e.g., XY-scanner 28 and fast-Z scanner 25). An advantage of the “fast-scan-slow-sweep” scanning scheme is that it only uses small field optics (e.g., a field diameter of 1.5 mm) which can achieve high focus quality at relatively low cost. The large surgical field (e.g., a field diameter of 10 mm or greater) is achieved with the XY-scanner, which may be unlimited.

In the laser system described above, due to the nature of the resonant scanning motion, the mirror angle as a function of time is a sinusoidal function. Thus, at or near the turning points, the scanning speed is zero or close to zero. As the spot-to-spot separation of the scanned laser focus spots within the eye tissue is proportional to the scanning speed, the laser focus spots can be close to each other and overlap near the turning points at the two ends of the scanline. Such high spatial density of laser focus spots in the eye tissue may cause excessive gas bubbles generated in the tissue, resulting in undesirable opaque bubble layers, and redundant laser pulse deposition in the eye.

To solve this problem, embodiments of the present invention provide systems and methods to eliminate the closely spaced or overlapping laser focus spots resulting from the resonant scanner used in a femtosecond laser system. This improvement can improve the spatial uniformity of laser spot distribution, reduce the undesirable opaque bubble layer formation, excessive gas bubbles, and the redundant laser pulse deposition during ophthalmic surgeries, such as corneal refractive surgery. Preferred embodiments of the present invention provide a system and method to physically block the laser spots near the two ends of the resonant scanline.

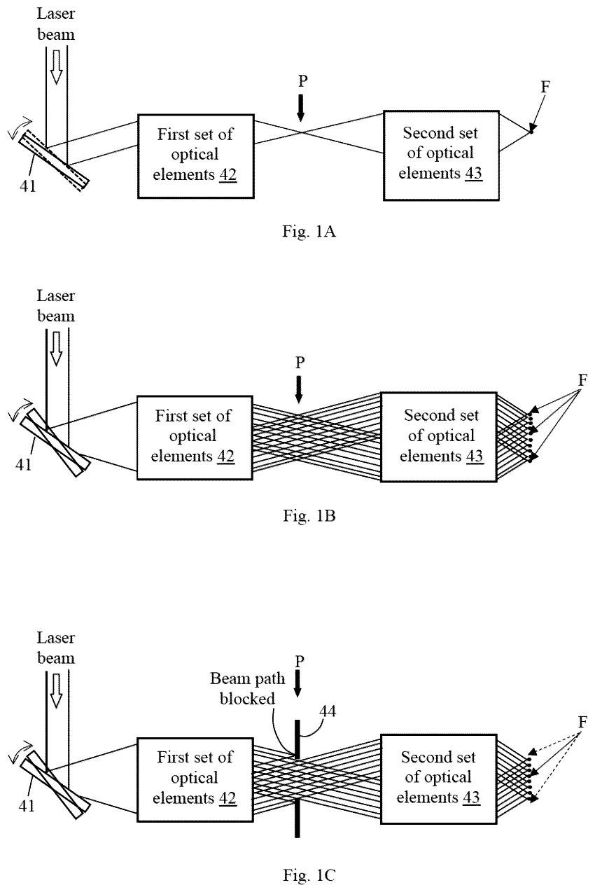

FIGS. 1A-1C schematically illustrate a portion of an optical system of an ophthalmic laser system and the principle of scanline truncation (blocking). FIG. 1A shows the relevant laser beam path corresponding to a specific rotation angle of the resonant scanning mirror (the laser device is not shown). The portion of the optical system includes a resonant scanning mirror 41 (corresponding to the resonant scanner 21 in FIG. 7), a first set of optical elements 42, and a second set of optical elements 43. Each set of optical elements includes one or more lenses, and may include a part of the objective lens assembly of the laser system.

As shown in FIG. 1A, the incoming laser beam (a parallel beam) is reflected by the resonant scanning mirror 41 to a scanned beam at a particular angle, and focused by the first set of optical elements 42 to an internal focus point located on an internal focal plane P of the optical system. The divergent laser beam emanating from the internal focus point is focused by the second set of optical elements 43 to an external focus spot F, which is located within the eye tissue during eye surgery.

The first and second set of optical elements 42 and 43 may be located, for example, on the fast scanline movement control module 20 and/or the movable XY stage 28 in the laser system shown in FIG. 7. In one implementation, one or more lenses of the first set of optical elements 42 and one or more lenses of the second set of optical elements 42 form the beam expander of the fast scanline movement control module 20 to generate parallel light beams; another part of the second set of optical elements 43 includes the objective lens on the movable XY stage 28 which focuses the parallel beams to the external focus spots F.

FIG. 1B shows the laser beam paths for the full scanning range of the resonant scanning mirror 41, without beam blocking. Each scanned beam corresponding to a particular rotation angle of the resonant scanning mirror 41 is focused by the first set of optical elements 42 to an internal focus point on the internal focal plane P, and the divergent light beam after the internal focal plane P is focused by the second set of optical elements 43 to an external focus spot F in the eye tissue. As the resonant mirror 41 rotates back and forth within its scanning range, the scanned beams are focused through a plurality of internal focus points to a plurality of external focus spots F, forming a scanline in the eye tissue. The focus spot density is higher at the two ends of the scanline, which is not desired.

FIG. 1C shows the portion of the optical system of the ophthalmic laser system with a beam blocking member 44 placed on or in the vicinity of the internal focal plane P to block a peripheral area of the beam paths. Thus, while the resonant scanning mirror 41 still scans its full scanning angular range, beams located in the peripheral area of the internal focal plane P are blocked from passing through, so that the high-density laser focus spots at the two ends of the scanline are eliminated.

In preferred embodiments, the length of the scanline that is blocked is approximately 1-10% at each end of the scanline.

The location of the beam blocking member 44 should be located on or in the vicinity the internal focal plane. One important design consideration is to fully block the beam but without having too high laser power density on the beam blocking material to cause material damage. Thus, the beam blocking member may be located at a predefined small distance away (in the direction of the optical axis) from the internal focal plane P to reduce the power density of the laser beam incident on the beam blocking member. For example, the beam blocking member may be placed at a position where the laser focus spots formed by the first set of optical elements 42 on the beam blocking member are between 10 and 20 μm in diameter, such as approximately 15 μm.

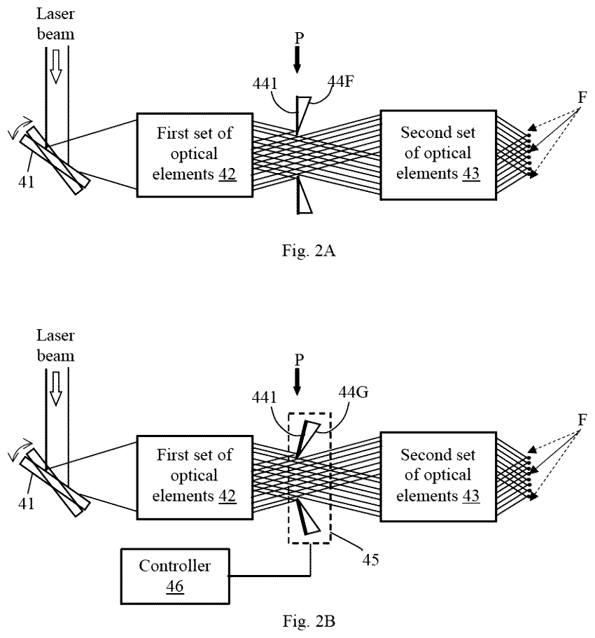

In a preferred embodiment, the beam blocking materials of the beam blocking member 44 is one that can endure high laser power density with long lifetime, such as ceramics and other high bandgap materials, or a bulk material with a high-power optical coating. Another consideration is that when blocking femtosecond laser pulses, the beam blocking materials should not deposit its own material to contaminate the other optics components. FIG. 2A schematically illustrates the portion of the optical system with a beam blocking member 44F, where the surface portion 441 that interacts with the incoming laser beam (the beam blocking surface) is coated with a high-power durable optical coating, such as multilayer dielectric glass coating with high reflection or silver metallic coating with high reflection or ceramic materials. The body of the beam blocking member 44F may be formed of glass or other suitable materials.

In some embodiments, the beam blocking member 44 is shaped and positioned such that the incident angle of the incoming laser beam on the beam blocking surface of the beam blocking member reduces the amount of reflected laser beam that goes back through the optical system to the laser device, which may cause laser instability. FIG. 2B schematically illustrates the portion of the optical system with a beam blocking member 44G, where the beam blocking surface 441 is disposed at an oblique (non-perpendicular) angle with respect to the optical axis of the first set of optical elements 42. The suitable angle may depend on other parameters of the optical system. In preferred examples, the beam blocking surface 441 is about 5 degrees or more with respect to the direction perpendicular to the optical axis.

Preferably, the cross-sectional shape of the beam blocking member 44 (in a cross-section that passes through the optical axis of the optical system) is such that the edges that block the laser beam are a tapered shape (wedge shape) ending at a knife edge, so as to effectively block the beam that is to be blocked and keep the other beams intact, as shown in FIGS. 2A and 2B (note that the coatings on the beam blocking surfaces are exaggerated in the drawings for emphasis). Note that the wedge shape is not required to be a thin wedge; it may have any wedge angle so long as the orientation of the non-beam-facing side of the beam blocking member avoids blocking any beam. In fact, a relatively thick wedge can be advantageous because it increases the heat capacity of the beam blocking portion.

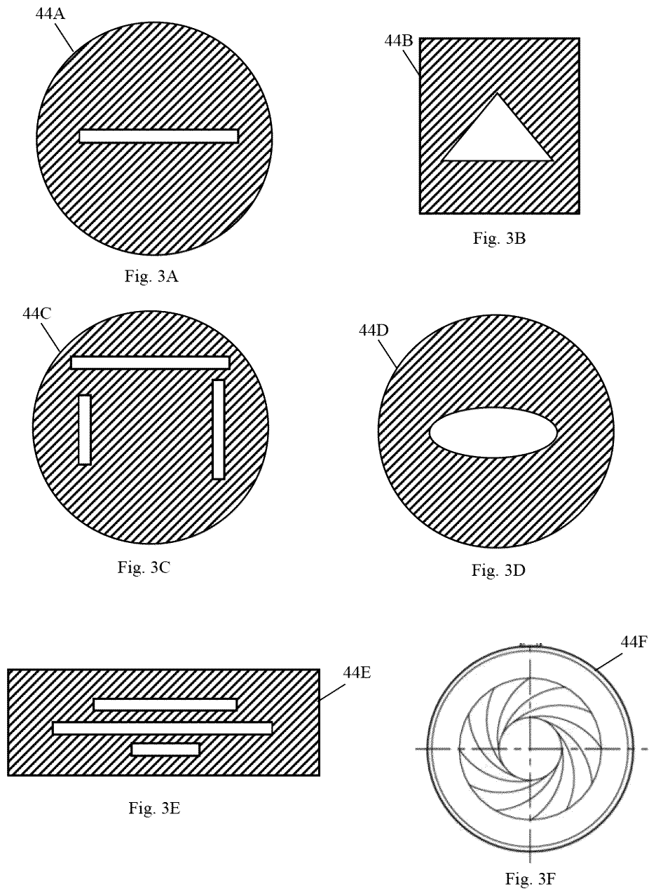

In some embodiments, the beam blocking member 44 is a plate (flat or curved) shape with one or more apertures. FIGS. 3A-3F illustrate some examples of the shape of the beam blocking member in the plan view, i.e., viewed along the direction of the optical axis. FIG. 3A shows a blocking member 44A with one rectangular aperture (a slit). This blocking member may be used to produce a scanline of a fixed length determined by the length of the slit (the laser scanline extends in the horizontal direction in this view). FIGS. 3C and 3E respectively show blocking members 44C and 44E with several rectangular apertures (slits) of different lengths, for producing scanlines with different lengths. The beam blocking member may be moved, including translated and/or rotated, to select different slits for blocking, thereby adjusting the length of the resulting laser scanline.

FIG. 3B shows blocking member 44B with a triangular aperture, forming a sliding variable aperture. This blocking member may be moved up and down to select the effective length of the aperture for blocking (the laser scanline extends in the horizontal direction in this view), thereby adjusting the length of the resulting laser scanline. The sliding variable aperture may alternatively have a trapezoidal shape or other non-rectangular shapes.

FIG. 3D shows blocking member 44D with an elliptical aperture, forming a rotating variable aperture. This blocking member may be positioned with the center of the aperture at the optical axis, and rotated around the optical axis to select the effective length of the aperture for blocking, thereby adjusting the length of the resulting laser scanline. The rotating variable aperture may alternatively have a polygonal shape or other non-circular shapes.

Each of the examples in FIGS. 3B-3E have fixed-size apertures where the blocking plate is moved as a whole to select the resulting scanline lengths.

FIG. 3F shows a beam blocking member 44F using an adjustable iris aperture similar to that in a camera. The adjustable iris aperture may be formed by multiple overlapping leaves surrounding the center of the aperture, where the leaves may be rotated to change the aperture size. The leaves may be coated with

The exemplary aperture shapes shown in FIGS. 3B-3F are non-limiting; apertures of other shapes, or combinations of the above shapes, may be used.

In the above examples, the inner edges of the apertures (at least the edges that are used to block the laser beam) are preferably tapered to form a knife edge in the cross-sectional view, such as those shown in FIGS. 2A and 2B. Further, the plate of the beam blocking member is preferably bent or curved so that the portions of the plate in the vicinity of the aperture edges, i.e. the portions that block the laser beam, form non-perpendicular angles with respect to the optical axis, such as that shown in FIG. 2B.

The above-described beam blocking members can provide adjustability to allow different lengths of laser scanlines to be formed, and can perform the adjustment within short transition periods (for example, less than 150 ms), so that the transition does not significantly slowdown the femtosecond laser surgery.

A mechanical support and movement structure 45 is provided to support and move the beam blocking member 44. The mechanical support and movement structure 45 may be implemented by any suitable structures, including, without limitation, one or more of linear actuators, stepping motors, support rails, gears, other suitable mechanical linkages, etc. When an adjustable iris aperture is used, the mechanical support and movement structure 45 is a part of the adjustable iris aperture assembly. The mechanical support and movement structure 45 is controlled by a controller (e.g., a computer, a microprocessor, etc.) of the ophthalmic laser system. The mechanical support and movement structure 45 and the controller 46 are schematically illustrated in FIG. 2B.

The controller controls the laser device 14, the resonant scanner 21, the scan line rotator 23, the mechanical support and movement structure 45, the XY-scanner 28, and the Z-scanner 25 and 27 in a synchronized manner according to programmed scan patterns to scan the laser scanline in the patient's eye to perform eye surgery.

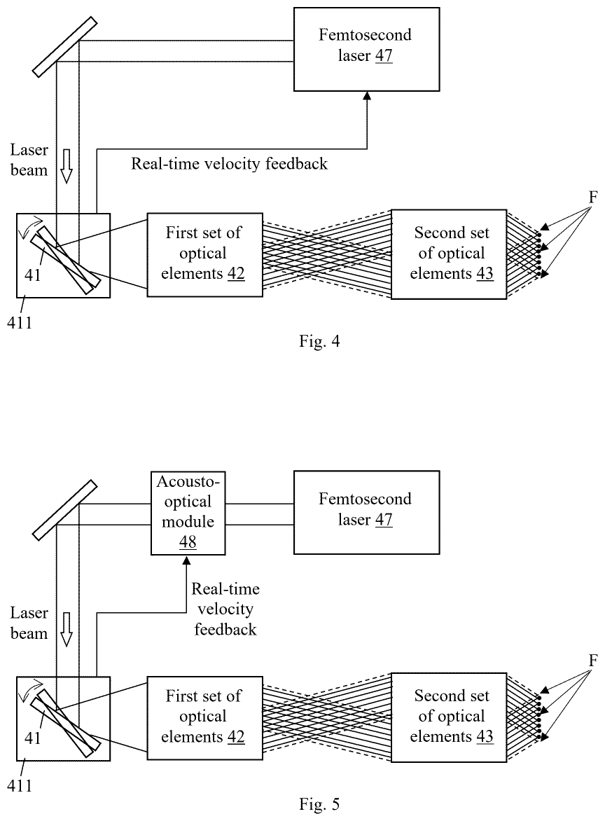

FIG. 4 schematically illustrates an alternative method to control photodisruption at the two ends of a scanline. In this method, a real-time feedback signal which indicates the velocity of the resonant scanner 41, provided by the driving electronic circuit 411 of the resonant scanner, is used to control the laser pulse repetition rate. This is achieved by controlling the pulse picker frequency in the laser control board of the laser device 47. To eliminate photodisruption near the two ends of the scanline, the laser repetition rate is increased to a predetermined high repetition rate within a defined time interval around the turning points of the scanline. In such a state, the laser generates laser pulses at a higher pulse repetition rate but a lower pulse energy. The lower pulse energy is such that at the locations they are delivered to the tissue, the pulse energy is below the tissue's photodisruption threshold (the energy at which the laser pulse starts to photodisrupt the tissue), and therefore will not result in any tissue cutting. This results in the elimination of photodisruption near the two ends of the scanline. Alternatively, the laser pulse repetition rate may be decreased to a predetermined low repetition rate within a defined time interval around the turning points of the scanline, so the number of pulses is reduced at the two ends of the scanlines. In FIG. 4, the laser beams at the two ends of the scanline are schematically shown in dashed lines to indicate the elimination or reduction of photodisruption.

The values of the predetermined high repetition rate and predetermined low repetition rate depend on characteristics of the laser device 47 and the optical system between the later and the eye, and may be determined by those skilled in the art using routine methods. The mechanism of adjusting the laser pulse repetition rate is also known in the art.

FIG. 5 schematically illustrates another alternative method to eliminate the laser pulses at the two ends of a scanline. In this method, the real-time feedback signal which indicates the velocity of the resonant scanner 41, provided by the driving electronic circuit 411 of the resonant scanner, is used to control an external pulse picker device (e.g. an acousto-optical module) 48 that can reject a laser pulse (e.g. to deflect it) or let the laser pulse pass through based on the resonant scanner's velocity. The pulse picker device is controlled to reject pulses within a defined time interval around the turning points of the scanline. In FIG. 5, the laser beams at the two ends of the scanline are schematically shown in dashed lines to indicate their absence.

It is noted that other parameters of the laser scanline may also be controlled and adjusted; such adjustments, coupled with scanline truncation described above, may achieve various additional desired results. For example, by increasing or decreasing the scanning amplitude of the resonant scanner, the scanline length and overall spot-to-spot separation may be increased or decreased before truncation. In such a case, by using a fixed truncation aperture, the truncated scanline length remains unchanged while the spot-to-spot separation is modified and optimized for tissue incision.

It will be apparent to those skilled in the art that various modification and variations can be made in the ophthalmic laser system of the present invention without departing from the spirit or scope of the invention. Thus, it is intended that the present invention cover modifications and variations that come within the scope of the appended claims and their equivalents.

1. An ophthalmic laser system comprising:
a laser device configured to generate a pulsed laser beam having a plurality of laser pulses;
a high frequency scanner configured to scan the laser beam back and forth at a predefined frequency to form a plurality of scanned laser beams at different angles;
a first set of optical elements and a second set of optical elements, each set of optical elements including one or more lenses, configured to focus the scanned laser beams through an internal focal plane located between the first and second sets of optical elements to a plurality of external focus spots which form a laser scanline;
a beam blocking member located in a vicinity of the internal focal plane, wherein the beam blocking member has a plate shape and defines one or more apertures; and
a mechanical support and movement structure configured to support and move the beam blocking member in a transverse direction perpendicular to an optical axis of the first and second set of optical elements,
wherein the one or more apertures of the beam blocking member are configured to block a portion of the plurality of scanned laser beams to eliminate a portion of the external focus spots at two ends of the laser scanline, and the beam blocking member is configured to be moved in the transverse direction to different positions to block different portions of the plurality of scanned laser beams.

2. The ophthalmic laser system of claim 1, wherein the beam blocking member is configured to be moved in the transverse direction to different positions to block 1-10% of the plurality of scanned laser beams.

3. The ophthalmic laser system of claim 1, wherein the beam blocking member is located at a distance from the internal focal plane such that laser focus spots formed by the first set of optical elements on the beam blocking member is between 10 and 20 μm in diameter.

4. The ophthalmic laser system of claim 1, wherein the beam blocking member is formed of a ceramic material or is coated with a high-power optical coating on a surface facing the first set of optical elements.

5. The ophthalmic laser system of claim 1, wherein a portion of a surface of the beam blocking member facing the first set of optical elements is disposed at a non-perpendicular angle with respect to the optical axis.

6. The ophthalmic laser system of claim 1, wherein one or more edges of the one or more apertures of the beam blocking member have a tapered shape ending at a knife edge.

7. The ophthalmic laser system of claim 1, wherein the one or more apertures are selected from a group consisting of: a plurality of linear slits having different lengths and arranged parallel to each other, a plurality of linear slits having different lengths and arranged non-parallel to each other, a polygonal shaped aperture, and an oval shaped aperture.

8. The ophthalmic laser system of claim 1, further comprising:
an XY-scanner configured to deflect the pulsed laser beam, the XY-scanner being separate from the high frequency scanner;
a Z-scanner configured to modify a depth of a focus of the pulsed laser beam; and
a controller configured to control the laser device, the high frequency scanner, the XY-scanner and the mechanical support and movement structure.

9. A method implemented in an ophthalmic laser system, comprising:
by a laser device, generating a pulsed laser beam having a plurality of laser pulses;
by a high frequency scanner, scanning the laser beam back and forth at a predefined frequency to form a plurality of scanned laser beams at different angles;
by a first set of optical elements and a second set of optical elements, each set of optical elements including one or more lenses, focusing the scanned laser beams through an internal focal plane located between the first and second sets of optical elements to a plurality of external focus spots which form a laser scanline;
by a beam blocking member located in a vicinity of the internal focal plane, blocking a portion of the plurality of scanned laser beams to eliminate a portion of the external focus spots at two ends of the laser scanline, wherein the beam blocking member has a plate shape and defines one or more apertures; and
by a mechanical support and movement structure, supporting and moving the beam blocking member in a transverse direction perpendicular to an optical axis of the first and second set of optical elements to different positions to block different portions of the plurality of scanned laser beams.

10. The method of claim 9, wherein the beam blocking member is configured to be moved in the transverse direction to different positions to block 1-10% of the plurality of scanned laser beams.

11. The method of claim 9, wherein the beam blocking member is located at a distance from the internal focal plane such that laser focus spots formed by the first set of optical elements on the beam blocking member is between 10 and 20 μm in diameter.

12. The method of claim 9, wherein the beam blocking member is formed of a ceramic material or is coated with a high-power optical coating on a surface facing the first set of optical elements.

13. The method of claim 9, wherein a portion of a surface of the beam blocking member facing the first set of optical elements is disposed at a non-perpendicular angle with respect to the optical axis.

14. The method of claim 9, wherein one or more edges of the one or more apertures of the beam blocking member have a tapered shape ending at a knife edge.

15. The method of claim 9, wherein the one or more apertures are selected from a group consisting of: a plurality of linear slits having different lengths and arranged parallel to each other, a plurality of linear slits having different lengths and arranged non-parallel to each other, a polygonal shaped aperture, and an oval shaped aperture.

16. The method of claim 9, further comprising:
by an XY-scanner, deflecting the pulsed laser beam;
by a Z-scanner, modifying a depth of the plurality of external focus spots; and
by a controller, controlling the laser device, the high frequency scanner, the XY-scanner, the Z-scanner, and the mechanical support and movement structure in a synchronized manner.

17. An ophthalmic laser system comprising:
a laser device configured to generate a pulsed laser beam having a plurality of laser pulses;
a high frequency scanner configured to scan the laser beam back and forth at a predefined frequency to form a plurality of scanned laser beams at different angles;
a first set of optical elements and a second set of optical elements, each set of optical elements including one or more lenses, configured to focus the scanned laser beams through an internal focal plane located between the first and second sets of optical elements to a plurality of external focus spots which form a laser scanline; and
an adjustable iris aperture located in a vicinity of the internal focal plane, the adjustable iris aperture including a plurality of moveable leaves,
wherein the adjustable iris aperture is configured to block a portion of the plurality of scanned laser beams to eliminate a portion of the external focus spots at two ends of the laser scanline, and to change a size of the aperture to block different portions of the plurality of scanned laser beams.

18. The ophthalmic laser system of claim 17, wherein the size of the aperture to is configured to be adjusted to block 1-10% of the plurality of scanned laser beams.

19. The ophthalmic laser system of claim 17, wherein the moveable leaves located at a distance from the internal focal plane such that laser focus spots formed by the first set of optical elements on the adjustable iris aperture is between 10 and 20 μm in diameter.

20. The ophthalmic laser system of claim 17, wherein a portion of a surface of the adjustable iris aperture facing the first set of optical elements is disposed at a non-perpendicular angle with respect to the optical axis.
